(19) United States
(12) Patent Application Publication
(10) Pub. No.: US 2026/0335137 A1
(43) Pub. Date: Oct. 29, 2026

(54) SYSTEM AND METHOD FOR EXPOSING A RETINA OF A SUBJECT TO A POLARIZATION PROFILE

(71) Applicant: INCOHERENT VISION INC., Wellesley (CA)

(72) Inventors: Connor KAPAHI, Waterloo (CA); Dmitry PUSHIN, Toronto (CA); Dusan SARENAC, Waterloo (CA); Andrew SILVA, Kitchener (CA)

(21) Appl. No.: 19/476,815

(22) PCT Filed: Apr. 18, 2024
(86) PCT No.: PCT/CA2024/050502

§ 371 Date: Oct. 20, 2025

Related U.S. Application Data
(60) Provisional application No. 63/497,449, filed on Apr. 21, 2023.

(51) Int. Cl. A61F 9/08; H04N 23/56

(52) U.S. Cl. CPC A61F 9/08; H04N 23/56

(57) ABSTRACT

The process can include generating structured light having a polarization profile at a state-preparation plane; and imaging the polarization profile of the structured light from the state-preparation plane to a retina of a subject.

### BACKGROUND

Structured light having a polarization profile consisting of spatially and/or temporally varying polarizations of light can be propagated to a subject's retina to generate an entoptic profile at the subject. There are various ways of generating structured light. U.S. Pat. No. 11,564,562 describes several examples of structured light generators suitable for this purpose. While existing structured light generators were suitable to a certain degree, there always remains room for improvement. In particular, structured light is generated at a location which will be referred to herein as a plane. At any position in the beam beyond the plane where the structured light is prepared, the spatial profile will naturally be altered by the effects of beam propagation in free space. For example, in an embodiment described in U.S. Pat. No. 11,564,562, a dark central region obstruction develops over distance, which increases in size with the propagation of the structured light beam. Such factors may significantly hinder the ability to use the structured light in many embodiments.

### SUMMARY

It was found that rather than propagating the structured light to the subject's retina, the plane where the structured light is generated could be imaged onto the subject's retina, which can avoid or attenuate the alterations in the spatial profile which would otherwise occur due to propagation between the plane and the user's retina.

In accordance with a first aspect of the present disclosure, there is provided a method comprising: generating structured light having a polarization profile at a state-preparation plane; and imaging the polarization profile of the structured light from the state-preparation plane to a retina of a subject.

Further in accordance with the first aspect of the present disclosure, the polarization profile can for example be a first polarization profile, the method can for example further comprise: at a computer, collecting feedback from the subject following said imaging, selecting a second polarization profile based on said collected feedback, and repeating the steps of generating and imaging with the second polarization profile.

Still further in accordance with the first aspect of the present disclosure, the method can for example further comprise repeating the steps of generating, imaging, collecting, and selecting over a plurality of iterations, for a plurality of different polarization profiles, where a value of a parameter of the different polarization profiles changes from iteration to the next, including, at each repeating, comparing the subject's feedback to the subject's feedback from the previous iteration, until the comparison yields a change.

Still further in accordance with the first aspect of the present disclosure, the method can for example further comprise, when the comparison yields a change, outputting a current value of the parameter as a limit value of the parameter for the subject.

Still further in accordance with the first aspect of the present disclosure, the different values of the parameter associated to different iterations can for example increase or decrease from one iteration to the next, the method can for example further comprise, when the comparison yields a change, changing the value of the parameter to the other one of said increase or decrease, and resuming said repeating.

Still further in accordance with the first aspect of the present disclosure, the method can for example further comprise outputting a current value of the parameter as a limit value of the parameter for the subject when the iterations are deemed to have converged.

Still further in accordance with the first aspect of the present disclosure, the method can for example further comprise outputting a current value of the parameter as a contrast sensitivity value of the parameter for the subject when the iterations are deemed to have converged.

Still further in accordance with the first aspect of the present disclosure, the parameter can for example be one of size of obstruction, form of obstruction, number of lines, density of azimuthal lines, rotation speed, and degree of polarization.

Still further in accordance with the first aspect of the present disclosure, said generating structured light can for example include generating a fixation point.

Still further in accordance with the first aspect of the present disclosure, the polarization profile can for example include at least three lines, preferably between 10 and 30 lines.

Still further in accordance with the first aspect of the present disclosure, the lines can for example be azimuthally distributed.

Still further in accordance with the first aspect of the present disclosure, said generating structured light can for example include rotating the polarization profile over time.

Still further in accordance with the first aspect of the present disclosure, the method can for example further comprise acquiring an image of the image of the polarization profile at the retina.

Still further in accordance with the first aspect of the present disclosure, the method can for example further comprise, exposing the retina of the subject to light having an intensity profile, the intensity profile having a geometrical correlation with the polarization profile.

In accordance with a second aspect of the present disclosure, there is provided a system comprising: a structured light generator configured for generating structured light having a polarization profile at a state-preparation plane; and an ocular imaging assembly configured for imaging the polarization profile from the state-preparation plane to a retina of a subject.

Further in accordance with the second aspect of the present disclosure, the system can for example further comprise a camera optically coupled to the retina via the ocular imaging assembly.

All technical implementation details and advantages described with respect to a particular aspect of the present invention are self-evidently mutatis mutandis applicable for all other aspects of the present invention.

Many further features and combinations thereof concerning the present improvements will appear to those skilled in the art following a reading of the instant disclosure.

### DESCRIPTION OF THE FIGURES

In the figures,

FIG. 1 is a block diagram of a system which can be used as a tool to assess a retina of a subject, in accordance with one or more embodiments;

FIG. 2 is an example of a system which can be used as a tool to assess a retina of a subject, in accordance with one or more embodiments;

FIG. 3a is a visual representation of first example of a polarization profile generated at a state-preparation plane, with FIG. 3b representing the associated entoptic profile perceived by a subject, in accordance with one or more embodiments;

FIG. 4a is a visual representation of second example of a polarization profile generated at a state-preparation plane, with FIG. 4b representing the associated entoptic profile perceived by a subject, in accordance with one or more embodiments;

FIG. 5 presents results of an iterative method for different participants, in accordance with one or more embodiments;

FIG. 6 presents results of an iterative method comparing a) a healthy participant and b) an AMD participant, in accordance with one or more embodiments;

FIG. 7 shows the average threshold visual angle radius across all participants for the OAM state stimuli. Experimental results are fit to expected threshold radii for each stimuli using a spatiotemporal energy model. Parameters for this model include the macular pigment optical density (MPOD) maximum amplitude, A₁, MPOD distribution width, ρ₁, and the threshold spatiotemporal energy value, Eₜ, in accordance with one or more embodiments;

FIG. 8 presents results for varying values of obstruction size, in accordance with one or more embodiments;

FIG. 9 is a flowchart of an example method, in accordance with one or more embodiments; and

FIG. 10 is an example of a computer, in accordance with one or more embodiments.

### DETAILED DESCRIPTION

The macula is the central region of the retina in the human eye, responsible for the highest fidelity image generation. Macular degeneration (MD), also known as age-related macular degeneration (AMD), is a debilitating eye disease that causes a loss of central vision. AMD is the global leading cause of blindness among people over the age of 60. Currently, there are no treatment options that can reverse vision loss due to macular degeneration; however, several treatment methods have been shown effective at slowing the progression of AMD and preventing the onset of AMD symptoms.

It was found that a method to characterize the macular profile and health is by testing the person's ability to perceive entoptic profiles induced by structured light stimuli (the structured light has a polarization profile, which, when correctly perceived by a subject's vision, can allow the subject to perceive an entoptic profile corresponding to the polarization profile). The physiological mechanism underlying the human ability to perceive polarized light is believed to lie within axons located in Henle's fiber layer of the retina that contains pigment molecules, such as lutein, with an average orientation radial to the central axis of the fiber. The dichroic properties of lutein lead to Henle's fiber layer acting like a radially oriented dichroic optical element in the central point of vision. Therefore, when a person with a healthy macula observes uniformly polarized light they can perceive a faint entoptic phenomenon consisting of two azimuthal fringes that is known as the Haidinger's brush. The structured light stimuli can be tailored to induce various strong entoptic signals that are visible and easily recognizable to a healthy macula, for example consisting of 20 azimuthal fringes. By testing the person's ability to perceive various stimuli that are projected to various locations on the retina, we can determine the size, health, and profile of their macula.

This can be achieved via a method including several steps. First, a structured light state that manifests a spatially-dependent polarity profile is prepared by a structured light generator. The polarity profile can have variable parameters such as: radial, azimuthal, vertical, and horizontal wave numbers; speed of entoptic profile motion; obstruction form and size. Second, the structured light can be imaged from the plane at which it is prepared at the structured light generator to the location of the subject's retina in a manner to subject the subject to the perception of entoptic profiles. The imaging of the structured light generation plane, as opposed to the propagation of the structured light from the plane to the subject's retina, can avoid propagation effects. Thirdly, feedback can be collected from the subject. Based on the feedback from the subject, one or more threshold values for parameters of the polarization profile can be collected. When a plurality of threshold values have been acquired for different parameters, it becomes possible to compute, with a reasonable degree of accuracy, higher-level data concerning the subject's physiology such as a spatial profile of macular pigment density or a spatial profile of the Henle fiber layer.

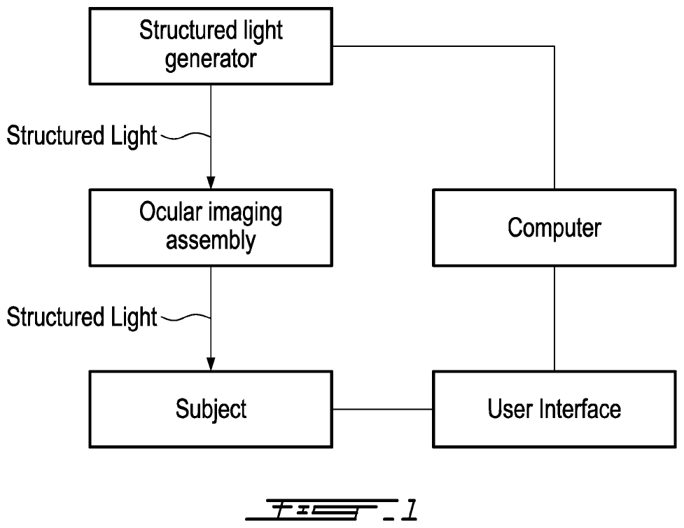

The method can be implemented via a system such as presented in FIG. 1, and which may include a structured light generator configured for generating structured light having a polarity profile at a state-preparation plane. The system can further include an ocular imaging assembly configured for imaging the polarity profile from the state-preparation plane to a retina of a subject. Various alternate embodiments of structured light generators are possible, some of which are described in U.S. Pat. No. 11,564,562, and additional examples of which are discussed below. Various alternate embodiments of ocular imaging assemblies are possible, examples of which can be based on optical systems such as microscopes and telescopes, including single lens, two-lens, 6 lens, 8 lens assemblies, to name a few examples. The ocular imaging assembly can be based on taking the real intensity, and may or may not apply magnification. By “ocular”, what is meant is an imaging assembly which is adapted to be used by a person.

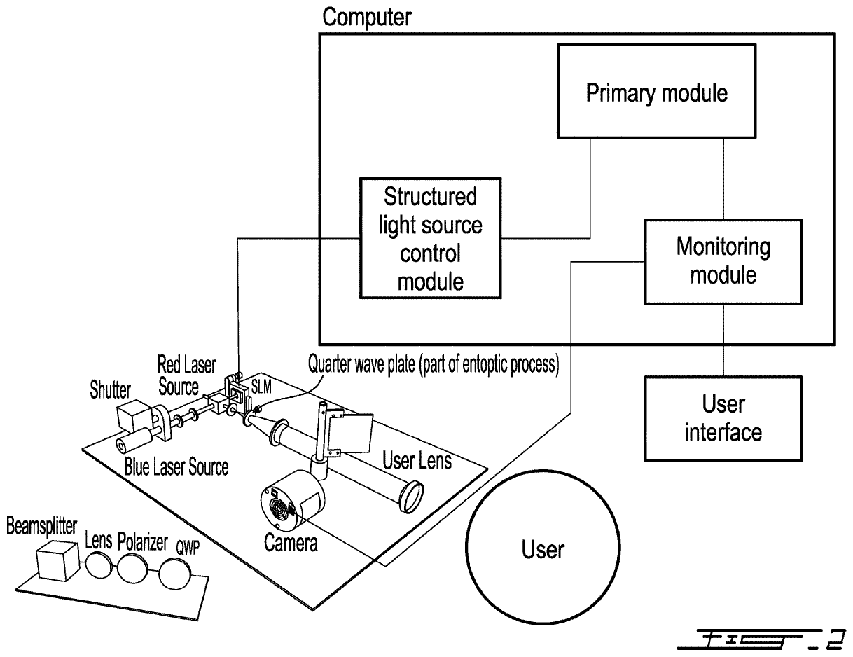

FIG. 2 presents an example embodiment of an optical diagnosis tool which can be used to perform a process such as presented above. The embodiment includes a number of components forming a structured light generator, and a number of components forming an ocular imaging assembly. It will be noted that in the illustrated embodiments, the components of the two sub-systems are intertwined along the optical axis, with a quarter wave plate of the structured light generator being position between two lenses of the ocular imaging assembly. This example shows that the two subsystems may not be completely distinct from one another.

In this example, the system includes a structured light generator in the form of a spatial light modulator (SLM), which can induce a controlled phase profile (polarity profile) over an incoming beam at a state-preparation plane. The pixel size of the SLM sets the available resolution. The ocular imaging assembly is provided here in the form of a 4f imaging system in combination with a 20D Volk lens which images the polarity profile from the state-preparation plane, coinciding here with the SLM, onto the participant's retina, without generating diffraction and/or other propagation effects. A small aperture illuminated by a red light is present in the middle of the stimulus to serve as a fixation point.

As shown in the figure, several elements of the system can be controlled by a controller, which can be provided in the form of a computer having a number of modules performing different software functions. In some embodiments, some of the modules or functions can be distributed rather than being centralized in a single computer. In particular, the SLM can be controlled by a structured light source control module which can, based on a request from a primary module, control the SLM to generate a particular structured light state.

For instance, consider the preparation of the following structured light state:

$$\vert Ψ 〉 = \frac{1}{\sqrt{2}} ( 1 - \prod [ r / c ] ) ( {e}^{- i ( {n}_{r} r + ℓ φ + θ t )} \vert R 〉 + \vert L 〉 )$$

where (r, φ) are the transverse coordinates, nᵣ and l and the radial and OAM numbers, |L> and |R> are the right and left circularly polarized states, Π[r/c] is the unit pulse function that sets the size c of the central obstruction area, and θt is a time-varying phase shift that dictates the speed of the perceived radial motion.

### Setting

nᵣ=0,l=7

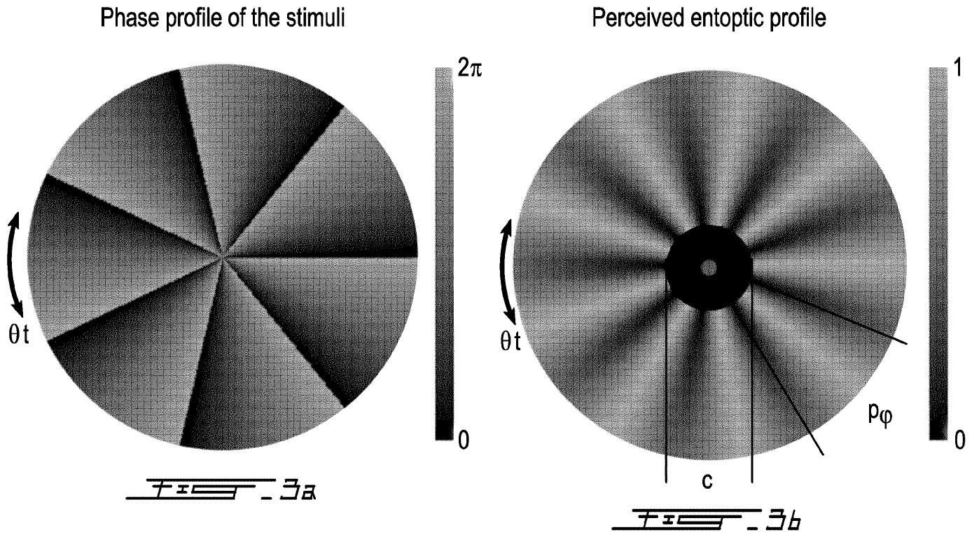

can produce, at the state-preparation plane, a polarization profile that consists of seven azimuthal fringes as seen in FIG. 3a, whereas a person may perceive an entoptic profile that consists of N=l+2=9 azimuthal fringes as shown in FIG. 3b.

### Whereas Setting

nᵣ=2π/pᵣ,l=2

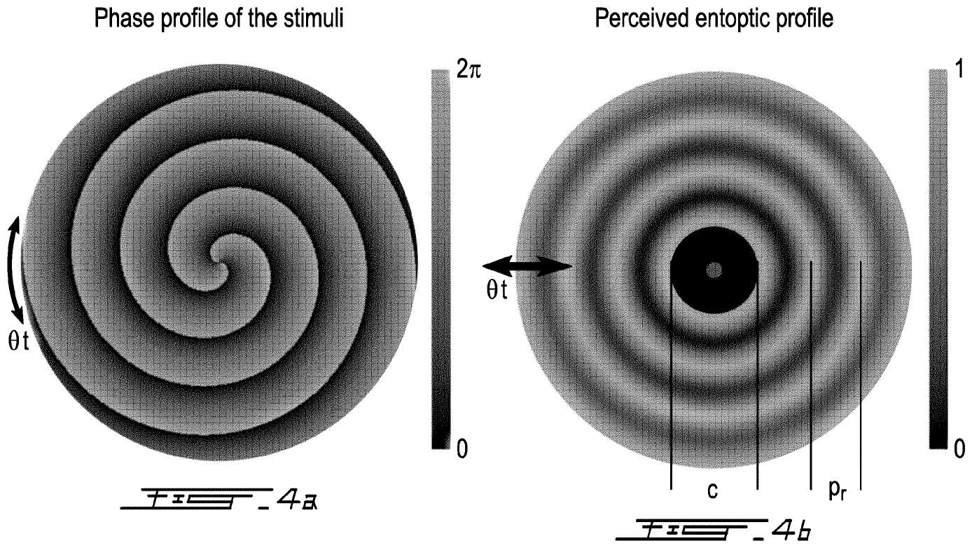

can produce, at the state-preparation plane, a spiral-like phase profile seen in FIG. 4a, whereas a subject may perceive the purely radial entoptic profile that is shown in FIG. 4b.

In the specific example presented in FIG. 2, monochromatic light from a 450 nm diode laser is prepared in a circularly polarized state, followed by a polarizer and quarter waveplate, each mounted on separate rotation stages. Following this, the beam is prepared in a polarization state which is orthogonal to the optic axis of the spatial light modulator (SLM). Light can then be prepared in a structured light state through the SLM. A two-lens telescope is used to expand the beam for viewing through a Volk 20D Binocular Indirect Ophthalmoscopy lens. As will be discussed further below, a translation stage mounted plate beamsplitter is placed before the Volk lens to provide for direct imaging of the retina while illuminated with the imaged structured light.

It was found that in some embodiments, features of the entoptic profile as perceived by the subject can depend on variables such as the individual's corneal birefringence (e.g. corneal and retinal thickness) and may need to be compensated for to achieve stable results when switching from one individual to another. It was found that when the number of lines in the polarization profile was above 3, while remaining discernable to the user, can produce a stimuli which is not dependent on the individual's corneal birefringence, and which therefor does not need to be compensated for this variable. Accordingly, it can be preferred for the polarization profile to have at least three azimuthally distributed lines. The maximum number of lines discernable by a user can depend on factors such as the size of the pixels in the polarization profile (e.g. the size of the pixels in the SML device in the example presented above), and the subject's vision. It is likely that a number of lines below 50 can be preferred. In some embodiments, it can be preferred for the number of lines to be between 10 and 30.

Let us consider an example where the polarization state is quantized along the circular polarization axis. The real-space coordinate system is defined with z following beam propagation, x on the horizontal, and y on the vertical with positive y pointing ‘up’. In this system, the diagonal state D is pointed in the positive x and y quadrant. Up to a normalization factor, these states are given by,

$$R = ( \begin{matrix}1 \\ 0\end{matrix} ) , L = ( \begin{matrix}0 \\ 1\end{matrix} ) ,$$

$$H = ( \begin{matrix}1 \\ 1\end{matrix} ) , V = ( \begin{matrix}1 \\ - 1\end{matrix} ) ,$$

$$D = ( \begin{matrix}1 \\ i\end{matrix} ) , A = ( \begin{matrix}1 \\ - i\end{matrix} ) .$$

Incident to a rotating polarizer, the laser is prepared in ψ₀=R state. Following the rotating polarizer, the state is described by,

ψ₁=R+e^{i2θ}L,

where the angle θ is defined relative to the horizontal axis in real space. Following the rotating polarizer, a rotating quarter-waveplate is set to either the diagonal or anti-diagonal axis. This produces one of two states,

ψ_{2,D}=H+e^{i2θ}v,

=cos(θ)R−sin(θ)L,

ψ_{2,A}=V+e^{i2θ}H,

=cos(θ)R+sin(θ)L,

where the waveplate setting has been denoted with the subscript D or A. The spatial light modulator will then write a spatially dependent phase on the horizontal polarization state, for example, we will choose an orbital angular momentum (OAM) state with OAM number,

ψ_{3,D}=e^{-ilφ}H+e^{i2θ}V,

ψ_{3,A}=e^{-ilφ}e^{i2θ}H+V.

The spatial light modulator is followed by a fixed quarter waveplate which is set to diagonal, mapping the states H→L and V→R. This produces one of two states,

ψ_{4,D}=e^{i2θ}R+e^{-ilφ}L,

ψ_{4,A}=R+e^{-ilφ}e^{i2θ}L.

The pattern perceived by a test subject can be modeled using a radial polarization filter. Assuming the macula perfectly polarizes, ie. D(r)=1, this filter produces an intensity pattern, given by,

$${I}_{D} = \frac{1}{4} ( {e}^{- i 2 θ} {e}^{i ℓ ϕ} ) ( \begin{matrix}1 & {e}^{- i 2 θ} \\ {e}^{i 2 θ} & 1\end{matrix} ) ( \begin{matrix}{e}^{i 2 θ} \\ {e}^{i ℓ ϕ}\end{matrix} ) ,$$

$${I}_{D} = \frac{1}{4} ( 2 + {e}^{- i 2 θ} {e}^{i ( ℓ + 2 ) ϕ} + {e}^{i 2 θ} {e}^{i ( ℓ + 2 ) ϕ} ) ,$$

$$= {cos}^{2} ( \frac{ℓ + 2}{2} ϕ + θ ) ,$$

$${I}_{A} = \frac{1}{4} ( 1 {e}^{- i 2 θ} {e}^{i ℓ ϕ} ) ( \begin{matrix}1 & {e}^{- i 2 θ} \\ {e}^{i 2 θ} & 1\end{matrix} ) ( \begin{matrix}1 \\ {e}^{- i ℓ ϕ} {e}^{i 2 θ}\end{matrix} ) ,$$

$${I}_{A} = \frac{1}{4} ( 2 + {e}^{i 2 θ} {e}^{- i ( ℓ + 2 ) ϕ} + {e}^{- i 2 θ} {e}^{i ( ℓ + 2 ) ϕ} ) ,$$

$$= {cos}^{2} ( \frac{ℓ + 2}{2} ϕ + θ ) .$$

The above shows that, for any l value, this state produces azimuthal fringes which rotate clockwise or counter-clockwise depending on the rotation waveplate setting. The effect of corneal birefringence can be considered by adding another waveplate element before the macular polarizer. Consider the strongest possible corneal effect, namely a half-waveplate oriented horizontally. The effect of this element is to map the states R→L and L→R.

$${I}_{D}^{′} = \frac{1}{4} ( {e}^{i ℓ ϕ} {e}^{- i 2 θ} ) ( \begin{matrix}1 & {e}^{- i 2 θ} \\ {e}^{i 2 θ} & 1\end{matrix} ) ( \begin{matrix}{e}^{- i ℓ ϕ} \\ {e}^{i 2 θ}\end{matrix} ) ,$$

$${I}_{D}^{′} = \frac{1}{4} ( 2 + {e}^{i 2 θ} {e}^{i ( ℓ - 2 ) ϕ} + {e}^{- i 2 θ} {e}^{- i ( ℓ + 2 ) ϕ} ) ,$$

$$= {sin}^{2} ( \frac{ℓ - 2}{2} ϕ + θ ) ,$$

$${I}_{A}^{′} = \frac{1}{4} ( {e}^{i 2 θ} {e}^{i ℓ ϕ} 1 ) ( \begin{matrix}1 & {e}^{- i 2 θ} \\ {e}^{- i 2 θ} & 1\end{matrix} ) ( \begin{matrix}{e}^{- i ℓ ϕ} {e}^{i 2 θ} \\ 1\end{matrix} ) ,$$

$${I}_{A}^{′} = \frac{1}{4} ( 2 + {e}^{- i 2 θ} {e}^{i ( ℓ - 2 ) ϕ} + {e}^{i 2 θ} {e}^{- i ( ℓ + 2 ) ϕ} ) ,$$

$$= {sin}^{2} ( \frac{ℓ - 2}{2} ϕ - θ ) .$$

Comparing these two cases with l=5, a participant with a corneal birefringence of γ_{cornea}=180° will see 3 azimuthal fringes while a participant with no corneal birefringence will see 5 lines. Critically, both participants will observe rotation clockwise or counter-clockwise at the same waveplate setting. In the case of a uniform polarization state, producing a Haidinger's brush entoptic pattern, the participants will observe,

I_{D}=cos²(φ+θ),I_{A}=cos²(φ−θ),

I′_{D}=cos²(φ−θ),I′_{A}=cos²(φ+θ),

thus for the same waveplate setting, each participant will observe an opposite rotation direction.

Assuming a horizontal orientation for the birefringence inherent in the cornea, we can generalize the above result for any rotation about the horizontal polarization by an angle, B. The effect of the cornea is given by the operation,

$${\hat{U}}_{C} = [ \begin{matrix}cos ( \frac{β}{2} ) & - i sin ( \frac{β}{2} ) \\ sin ( \frac{β}{2} ) & cos ( \frac{β}{2} ) .\end{matrix} ]$$

The state after the cornea is given by,

ψ_{S,D}=Û_{C}ψ_{4,D},

=(e^{i2θ} cos(β/2)−ie^{-ilφ} sin(β/2))R

+(ie^{i2θ} sin(β/2)−e^{-ilφ} cos(β/2))L,

ψ_{S,A}=Û_{C}ψ_{4,A},

=(cos(β/2)−ie^{i2θ}e^{-ilφ} sin(β/2))R

+(i sin(β/2)+e^{i2θ}e^{-ilφ} cos(β/2))L.

We can calculate the intensity profile for this generalized case using this state ψ_{S,D} or ψ_{S,A},

I_{D}=|Mψ_{S,D}|²

=½+½ cos([l+2]φ+2θ)cos²(β/2)

−½ sin(β/2)cos(β/2)sin(lφ+2θ)

−½ sin(β/2)cos(β/2)sin(2φ)

−½ cos([l−2]φ+2θ)sin²(β/2),

I_{A}=|Mψ_{S,A}|

=½+½ cos([l+2]φ−20)cos²(β/2)

−½ sin(β/2)cos(β/2)sin(lφ−2θ)

−½ sin(β/2)cos(β/2)sin(2φ)

−½ cos([l−2]φ−2θ)sin²(β/2).

While these patterns do not have a well-defined number of equal contrast fringes, the phenomenon seen in the previous case holds. That is, for OAM states with |l|>3, the entoptic pattern caused by the macula appears to rotate clockwise/counter-clockwise for every participant regardless of their corneal birefringence value. For the case of l=0, there exists a second birefringence value of interest, β=90°, where no continuous motion of Haidinger's brush is apparent.

Accordingly, the dependence to the ocular birefringence can be removed by choosing to use specific forms of structured states of light, for example a polarization-coupled OAM state shown in FIG. 3, as the direction of motion of the entoptic patterns generated by these states will not depend on corneal birefringence. More specifically, in the example of FIG. 3, the spatially dependent phase profile (polarization profile) of the stimuli and the simulated entoptic pattern that would be perceived by a participant with a healthy macula. A radial blur is applied to depict the effect of decreasing macular pigment density along the radial direction. The clockwise/counter clockwise rotation of the beam in a) results in a clockwise/counter clockwise motion of the perceived pattern. The experimental stimulus can include a central red light fixation guide, as shown. Finally, the size of the centrally blocked region, depicted in black, can be varied to control the difficulty of the task and measure the peripheral extent of sensitivity to azimuthal entoptic motion.

In the example shown in FIG. 4 the spatially dependent phase profile of the stimuli and the simulated entoptic pattern that would be perceived by a participant with a healthy macula are visually represented. The radial filter in the eye removes the spiral feature of the stimuli producing purely radial waves. A radial blur is applied to depict the effect of decreasing macular pigment density along the radial direction. The clockwise/counter clockwise rotation of the beam in a) results in an inward/outward motion of the perceived radial pattern. The experimental stimulus included a central red light fixation guide, as shown. Finally, the size of the centrally blocked region, depicted in black, can be varied to control the difficulty of the task and measure the peripheral extent of sensitivity to radial entoptic motion.

A method can be used to characterize the macular structure of the human eye using a psychophysical task that involves perceiving and discriminating various forms of structured light states, where the structured light states manifest a spatially and temporarily varying structured light state, and in which the polarization profile can be changed iteratively based on feedback from the subject in a manner to allow to determine one or more values of parameters associated to the subject's eyesight.

Indeed, the structured light state created by the structured light generator can be modified automatically, via a computer, based on subject response. This allow for the value of one or more parameter of the polarization profile, such as size of obstruction, form of obstruction, number of lines, density of azimuthal lines, rotation speed, etc., to be changed from one iteration to another, and based on subject response.

For instance, an indication can be acquired from the subject indicating whether the entoptic profile generated with the current polarization profile, associated to a current value of a parameter, is perceived more or less than the entropic profile which was generated by the former polarization profile, associated to a previous value of the parameter. The goal of the iterative process may be to determine the value of the parameter associated to the user. The value may be a threshold value, such as the limit value for the parameter at which the subject ceases to perceive a given feature of the entoptic profile (such as the entoptic profile as a whole). Accordingly, the value of the parameter may be increased or decreased from a starting value until the feedback collected from the subject indicates that the subject has ceased to perceive the given feature of the entoptic profile. In some embodiments, the current value of the parameter in the polarization profile can be outputted as the limit value associated to the subject. In an alternate embodiment, an iterative process can be conducted, and when the feedback collected from the subject indicates that the subject has ceased to perceive the given feature, rather than outputting the current value as the limit value, the automated process can continue to iterate but by changing the value in the opposite direction. In other words, if the value was previously increasing, the value may be changed by decreasing it for the next iteration, and possibly for yet another iteration, until the feedback collected from the subject has reversed (e.g. that the subject now perceives the given feature of the entoptic profile anew), at which stage the process may stop, or continue iterating in the opposite direction, and alternatingly, until the process is deemed to have converged, at which point the current value of the parameter can be outputted as the limit value associated to the subject. In one example, the parameter can be the size of a central obstruction in the beam (shown as “c” in FIG. 3 and FIG. 4). Thus the procedure to determine the threshold of perception for a particular participant in terms of one or more parameter such as visual angle, contrast sensitivity, so on, can be fully automated and performed in a relatively short timespan as required for commercial ocular diagnostic tools.

In one example, participants can be tasked to discriminate the apparent motion of the entoptic pattern. Alternately, the task can involve discriminating between the number of azimuthal fringes. However, setting the task to be a discrimination between clockwise and counter clockwise where the number of azimuthal fringes is greater than 5, makes the task insensitive to ocular birefringence.

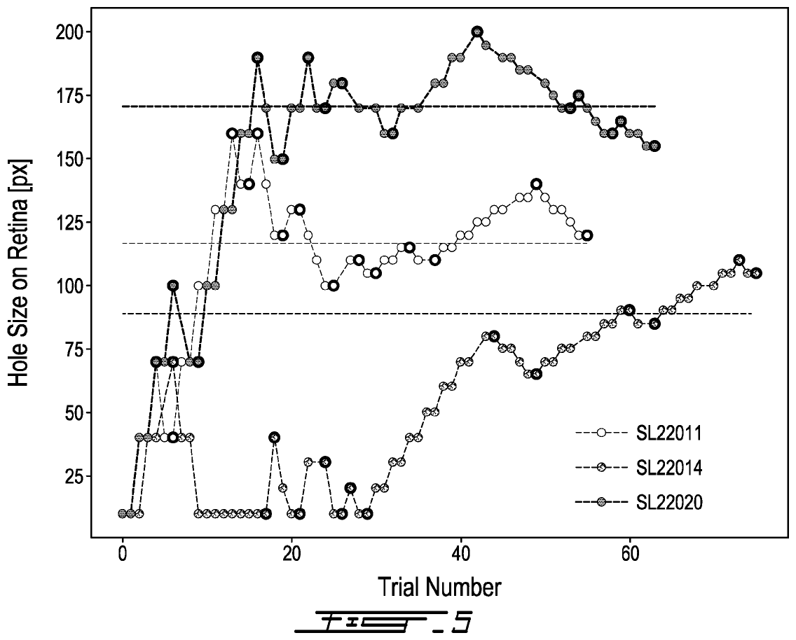

More specifically, the obstruction region c on the SLM can be varied according to an N-up, M-down psychophysical staircase. The obstruction region becomes larger after N consecutive correct responses and becomes smaller after every M incorrect response. FIG. 5 illustrates this procedure using a 2-up, 1-down staircase results for three different participants, with reversal points highlighted with a black outline. Threshold values from this 2-up, 1-down staircase, computed from the final six reversal points are shown as dotted lines. The results are quantified in the form of number of pixels. This procedure results in an obstruction size threshold indicating the eccentric extent of sufficient polarization sensitivity to achieve a targeted performance accuracy (70.7% performance accuracy in the case of a 2-up, 1-down procedure). A larger threshold indicates a larger eccentric range of polarization sensitivity.

Imaging the reflected structured light from the participant's retina can be used to translate the calculated results into real dimensions of degrees of visual angle and/or length (ie. millimeters). The thresholds can be converted into visual angle units through retinal imaging. Returning to FIG. 1, an example a camera is optically coupled, via the ocular imaging system, in a manner to acquire an image of the image of the polarization profile which is projected onto the retina by the ocular imaging system. The image is acquired from the retina. Retinal images using structured light illumination sources can then be used to measure the size of obstructions presented to the patient in real-space units, such as degrees of visual-angle or length on the retinal surface.

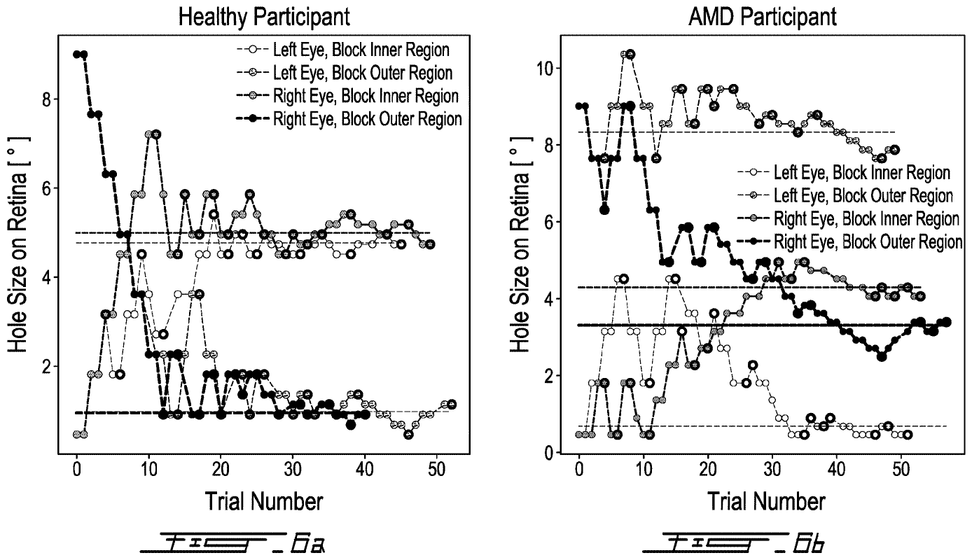

For instance, FIG. 6 presents example staircase results for a) a healthy patient and b) a patient with macular degeneration. Staircase results are shown for both left and right eyes, for OAM states with an obstruction that obstructs either the inner region, to a variable radius, or the outer region, to a variable radius. The radius of these obstructions is shown on the vertical axis. Patient performance on stimuli with an obstruction that blocks the outer region reaches a floor value for the healthy participant, while the result for the AMD patient does not. Furthermore, the AMD patient performance on identical stimuli varies significantly between eyes, while performance does not vary between eyes for the healthy patient. The results are plotted in real-space units, degrees of visual angle in this specific embodiment.

Various indicators which may be associated to macular health can be acquired. For instance, in one example, represented in FIG. 6, the evaluation of macular health can be performed through the measurement of patient performance on specific structured light stimuli that is indicative of a healthy macular structure. One possible structured light state which demonstrates a characteristic difference between healthy and unhealthy macular structures is an OAM state with an obstruction at radii greater than a value. FIG. 6 shows staircase results from two structured light stimuli in both eyes of a) a healthy patient and b) a patient with AMD. The two stimuli presented block either the inner region of the structured light stimulus or the outer region, each to a variable radius. Performance on both stimuli is identical between eyes for the healthy patient, while results for the AMD patient differ between eyes. Performance with stimuli which block the outer region reaches a minimum value for both eyes in the healthy patient, while the AMD patient shows threshold values above this minimum value in one eye.

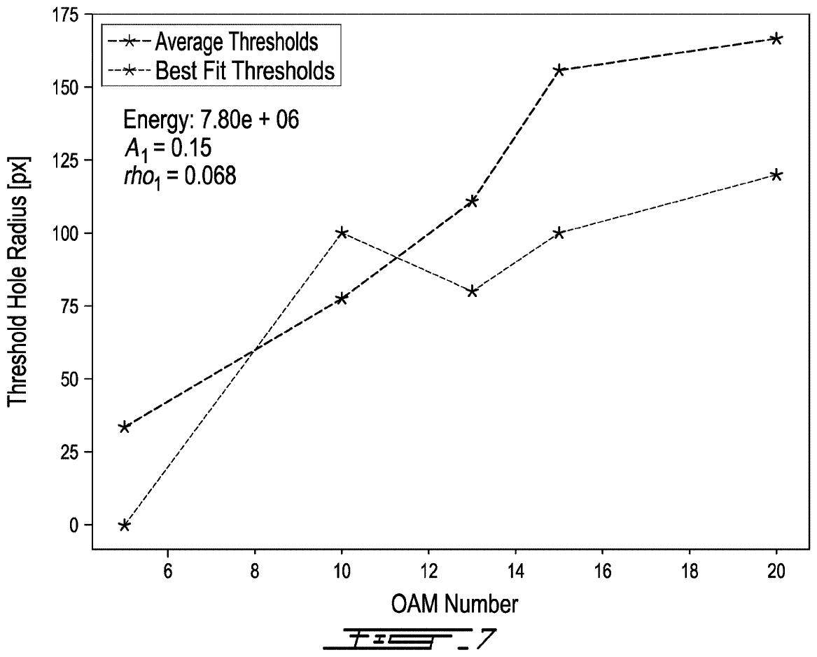

Measuring an individual's performance for various obstruction radii is one method for determining the apparent size of perceived entoptic patterns. FIG. 7 shows possible threshold perception results for states with various OAM number, l. Measurement of the obstruction size threshold at a specified success rate (or % correct) defines the apparent size of a particular entoptic pattern.

More elaborated indicators which may be associated to macular health can be acquired by performing additional iterations of the process in a manner to characterize values of additional parameters for the subject. Indeed, the entoptic patterns created by structured light profiles are caused by the macular pigments which are held in Henle's fiber layer of the macula. By presenting different structured light states (i.e., easier/harder to see), we can evaluate macular pigment density and Henle's fiber layer. These factors can be associated to macular health.

For instance, a method can be conducted to determine the apparent size of the perceived entoptic profiles. A patient's ability to see the entoptic patterns generated by structured light can be evaluated by moving the entoptic pattern (i.e. rotate clockwise/counter-clockwise) and testing if the patient can correctly detect the motion direction. The size of entoptic pattern a patient sees can be evaluated by using a central obstruction in conjunction with the motion detection task. At some threshold, the patient will no longer correctly perceive the motion direction. Examples of these thresholds can be seen in FIG. 7. This allows us to determine the spatial extend of their polarization-related entoptic vision

The method can be used to determine the spatial profile of macular pigment density in the retina of the human eye. Indeed, if you change the values of different parameters, you can compute what the profile is, e.g. different densities at different locations. This can be done by varying the values of several parameters such as the number of lines, different obstructions, different speeds of rotations, different radial fringe densities, finding the associated obstruction thresholds, etc.

Human vision is observed to respond with non-uniform sensitivity to stimuli of different spatial and temporal frequencies. The measurement of these sensitivities has been accomplished using, for the accepted standards, periodic stimuli of a constant spatial frequency in one direction (ie. a sinusoidal grating) which is made to move as a constant speed. These measurements form the basis for a class of perception models which model the response of the human eye to motion by convolving the 3-dimensional (2-space/1-time) motion of a stimuli with an appropriate spatiotemporal filter. The visibility of any stimuli can then be computed by integrating the result of this convolution across space and time, resulting in a single value called the spatiotemporal energy. As human vision is sensitive to a range of spatial and temporal motion frequencies, computing the spatiotemporal energy of a given stimuli requires the evaluation of multiple convolutions across three dimensions. Therefore, modern spatiotemporal energy analysis often make use of the Fourier transform of these stimuli, to simplify the computation.

In accordance with the example Fourier method, given an intensity pattern, I(x, y, t), we can compute the spatiotemporal energy, E, via

$$E = {\int }_{0}^{∞} d ω {\int }_{- ∞}^{∞} {dk}_{x} {\int }_{- ∞}^{∞} {dk}_{y} \vert ℱ [ I ( x , y , t ) ] S ( {k}_{x} , {k}_{y} , ω ) \vert ,$$

where F denotes the Fourier transform and (kₓ, k_{y}, ω) denote the Fourier conjugate of (x, y, t). Computing the 3D Fourier transform of this stimulus is difficult, but there are several options to achieve this. First, is using a 3D matrix of these intensity values and compute the FFT, experimentally this appears to take between 1-2 minutes. Another option is to compute the Fourier transform analytically.

To compute this analytically, consider the intensity profile created by an entoptic image. This should take the form,

$$I ( x , y , t ) = 1 - \frac{{e}^{- {r}^{2} ( x , y ) / {σ}^{2}}}{2} ( 1 + cos ( ℓ \cdot ϕ ( x , y ) + θ t ) ) ,$$

we can write this as a sum of three functions, and integrate each of these separately,

I⁽¹⁾(x,y,t)=1

$${I}^{( 2 )} ( x , y , t ) = \frac{{e}^{- {r}^{2} ( x , y ) / {σ}^{2}}}{2}$$

$${I}^{( 3 )} ( x , y , t ) = \frac{{e}^{- {r}^{2} ( x , y ) / {σ}^{2}} x}{2} cos ( ℓ \cdot ϕ ( x , y ) + θ t ) .$$

A significant parameter, not included in I(x, y, t), is the central obstruction of radius, Rₒ. This appears as a discontinuity at r=Rₒ, with I(r≤Rₒ, φ, t)=1. Including this, we can immediately determine,

F[I⁽¹⁾(x,y,t)]=δ(kₓ,k_{y},ω).

These equations are written in polar coordinates, therefore we can look to take the Fourier transform in polar coordinates as well,

$$ℱ [ I ( r , ϕ , t ) ] = {\int }_{- ∞}^{∞} d t {\int }_{0}^{∞} dr {\int }_{- π}^{π} d ϕ I ( x , y , t ) r \cdot {e}^{- i ( r {k}_{r} cos ( ϕ - {k}_{ϕ} ) + ω t )} .$$

Looking at the φ terms, it is reasonable to expect that this Fourier transform will take the form of Bessel functions of the first-kind, Jₙ(z), for n∈ [symbol] . While it is not obvious what the Fourier transform will be, we can decompose our function I(r, φ, t), using,

$$I ( r , ϕ , t ) = {\sum }_{n = - ∞}^{∞} {I}_{n} ( r , t ) {e}^{i n ϕ} ,$$

where

$${I}_{n} ( r , t ) = \frac{1}{2 π} {\int }_{- π}^{π} I ( r , ϕ , t ) {e}^{i n ϕ} d ϕ .$$

This is relevant because, looking back to our Fourier transformation in polar coordinates, we can now write the Fourier transform as,

$$ℱ [ I ( x , y , t ) ] = {\sum }_{n = - ∞}^{∞} {( - i )}^{n} {e}^{- i n {k}_{ϕ}} 2 π {\int }_{- ∞}^{∞} d t {\int }_{0}^{∞} dr {I}_{n} ( r , t ) {J}_{n} ( 2 π {k}_{r} r ) r \cdot {e}^{- i ω t} .$$

Since the second term has no φ dependence, the function I⁽²⁾(r, φ, t) is equal to Iₙ₌₀(r,t). Therefore,

$$ℱ [ {I}^{( 2 )} ( r , ϕ , ω ) ] = 2 π δ ( ω ) {\int }_{0}^{∞} dr {I}^{( 2 )} ( r ) {J}_{0} ( 2 π {k}_{r} r ) r . \qquad ( 11 )$$

The second term has no dependence on φ, nor on t, therefore we can reduce the Fourier transform to,

$$ℱ [ {I}^{( 2 )} ( r , ϕ , t ) ] = δ ( {k}_{ϕ} ) ( \frac{1}{2} {\int }_{{R}_{o}}^{∞} dr {e}^{- {r}^{2} ( x , y ) / {σ}^{2}} r \cdot {e}^{- i r {k}_{r}} + \frac{1}{2} {\int }_{0}^{{R}_{o}} dr r \cdot {e}^{- i r {k}_{r}} )$$

These integrals have a closed form solution. Note that this distribution is an example, let us look at these integrals using the correct distributions,

$$\frac{1}{2} {\int }_{{R}_{O}}^{∞} {re}^{- i r {k}_{r}} {A}_{1} 1 {0}^{- {ρ}_{{1}^{′}} r} dr = - \frac{{e}^{{R}_{o} ( {ρ}_{{1}^{′}} + i {k}_{r} )} ( i {k}_{r} {R}_{o} + {ρ}_{{1}^{′}} {R}_{o} + 1 )}{2 {( {k}_{r} - i {ρ}_{{1}^{′}} )}^{2}} ,$$

where ρ_{1′}=ρ₁ log 10. Next,

$$\frac{1}{2} {\int }_{0}^{{R}_{o}} {re}^{- i {k}_{r} r} dr = \frac{{e}^{- i {k}_{r} {R}_{o}} ( 1 - i {k}_{r} {R}_{o} ) - 1}{2 {k}_{r}^{2}}$$

Therefore the first terms, I⁽¹⁾ and I⁽²⁾, have closed form solutions, let us now consider the third term.

The Fourier transform for I⁽³⁾,

$$ℱ [ {I}^{( 3 )} ( x , y , t ) ] = {\int }_{- ∞}^{∞} d t {\int }_{0}^{∞} dr {\int }_{- π}^{π} dϕ \{ \begin{matrix}{e}^{- {r}^{2} ( x , y ) / {σ}^{2}} 2 & : r \geq {R}_{o} , \\ 1 & : r < {R}_{o}\end{matrix} \} cos ( ℓ \cdot ϕ ( x , y ) + θ t ) r \cdot {e}^{- i ( r {k}_{r} cos ( ϕ - {k}_{ϕ} ) + ω t )} ,$$

is not straightforward.

Let us look at the cosine term of the intensity pattern,

$${I}_{n}^{( 3 )} ( r , ϕ , t ) = \frac{1}{2 π} {\int }_{- π}^{π} \{ \begin{matrix}{e}^{- {r}^{2} / {σ}^{2}} 2 & : r \geq {R}_{o} , \\ 1 & : r < {R}_{o}\end{matrix} \} cos ( ℓ \cdot ϕ + θ t ) {e}^{in ϕ} d ϕ = \{ \begin{matrix}{e}^{- {r}^{2} / {σ}^{2}} 2 & : r \geq {R}_{o} , \\ 1 & : r < {R}_{o}\end{matrix} \} cos ( - θ t ) δ ( n - ℓ ) .$$

This delta function allows us to directly write the Fourier transform as,

$$ℱ [ {I}^{( 3 )} ( r , ϕ , t ) ] = ( {i}^{ℓ} {e}^{i ℓ {k}_{ϕ}} + {( - i )}^{ℓ} {e}^{- i ℓ {k}_{ϕ}} ) 2 π {\int }_{- ∞}^{∞} d t {\int }_{0}^{∞} dr {I}_{ℓ} ( r , t ) {J}_{ℓ} ( 2 π {k}_{r} r ) r \cdot {e}^{- i ω t} .$$

Integrating these Bessel functions is not straightforward for a general value, l, so let us first perform the integral over time,

$$ℱ [ {I}^{( 3 )} ( r , ϕ , t ) ] = ( {i}^{ℓ} {{e}^{i ℓ {k}_{ϕ}} ( - i )}^{ℓ} {e}^{- i ℓ {k}_{ϕ}} ) 2 π δ ( ω - θ ) {\int }_{0}^{∞} {rI}_{ℓ} ( r ) {J}_{ℓ} ( 2 π {k}_{r} r ) dr ,$$

where

$${I}_{ℓ} ( r ) = \{ \begin{matrix}{e}^{- {r}^{2} / {σ}^{2}} 2 & : r \geq {R}_{o} , \\ 1 & : r < {R}_{o}\end{matrix} \} .$$

This final integral over the radial coordinate does not have an analytical solution for every integer value, l. Therefore, we can evaluate this integral for each particular value of l that is of interest. This is possible analytically for Rₒ=0.

A second stimulus which is of interest is a series of concentric rings with period, a. This state is generated by an OAM state with l=2, and a radial term. The intensity profile of these states is given by,

$$I ( x , y , t ) = 1 - \frac{{e}^{- {r}^{2} ( x , y ) / {σ}^{2}}}{2} ( 1 + cos ( a \cdot r ( x , y ) + θ t ) ) ,$$

where it is clear that the only difference between this pattern and the entoptic pattern generated by an OAM state, is a sinusoidal intensity modulation in r instead of φ. Therefor the terms, I⁽¹⁾ and I⁽²⁾, are unchanged, while the third term, I⁽³⁾, requires a re-evaluation.

The dichroism of macular pigments, lutein in particular, is believed to produce a range of entoptic phenomenon when viewing monochromatic polarized light structures including Haidinger's Brush. Therefore, it is hypothesized that the contrast and radius of these macula induced entoptic phenomenon is directly correlated to macular pigment optical density. This presents the possibility of measuring macular pigment density through psychophysical experiments. Assuming that the macular pigment optical density (MPOD) follows,

D(r)=A₁10^{-ρ}^{₁}^{r},

we can reconstruct the entoptic image seen by an individual. Using this model, the threshold hole size can be computed assuming a threshold spatiotemporal energy value, Eₜ. the equation presented above can be used to compute the threshold hole size, Rₒ, for a particular energy threshold, Eₜ, macula parameters ρ₁ and A₁, and OAM number l. A preliminary fit of the measured hole-size for several OAM numbers suggests that the average MPOD and spatiotemporal energy parameters are Eₜ=7.80·10⁶, A₁=0.15, ρ₁=0.068(°)⁻¹.

The perceived entoptic pattern generated by a structured light state depends on the parameters of an individual's macular structure. An individual's macular structure is characterized by the thickness and size of an individual's Henle's fiber layer, the density of macular pigments at different locations on the retina, and the sensitivity of an individual's photoreceptors. One method of describing these factors is through the spatially varying macular pigment optical density (MPOD). One model of MPOD in humans is given by

D(r)=A₁10^{-ρ}^{₁}^{r}+A₂10^{-ρ}^{₂}^{(r-a}^{₂}⁾^{²},

where the amplitudes, A₁ and A₂, describe the amplitudes of the exponential and ring terms, respectively, the coefficients, ρ₁ and ρ₂, describe the characteristic size of the exponential and ring terms, respectively, the offset, α₂, describes the offset of the ring term, and r is the radial coordinate.

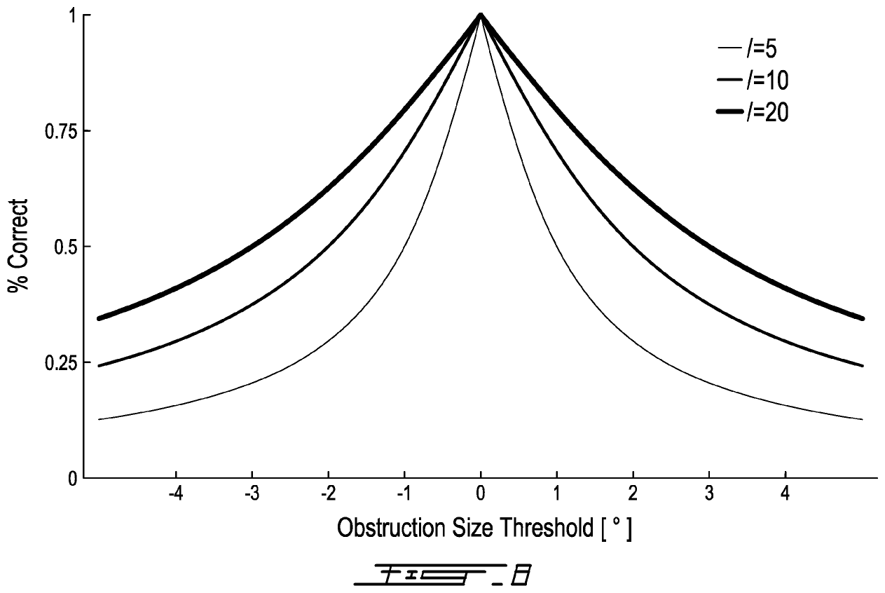

The strength or darkness of the entoptic pattern generated by structured light depends on the macular pigment density of the individual. Structured light stimuli can be generated which are easier/harder to see compared to other patterns. By finding the minimum perceivable pattern using a fixed radius central obstruction, the macular pigment density at that location can be measured. Repeating these measurements at several radii creates a spatial profile of an individual's macular pigment density, an example of which is shown in FIG. 8, where example success-rates as a function of central obstruction size for structured light states with different OAM numbers.

The method can be used to determine the spatial profile of the Henle fiber layer. This can be done via a mapping of macular pigment density or perhaps directly from results of characterization of values of different parameters for the subject. The clarity of entoptic pattern generated by structured light depends on the thickness of Henle's fiber layer, which contains macular pigments. By finding the minimum perceivable pattern using a fixed radius central obstruction, thickness of Henle's fiber layer at that location can be measured. Repeating these measurements at several radii creates a spatial profile of an individual's Henle's fiber layer thickness.

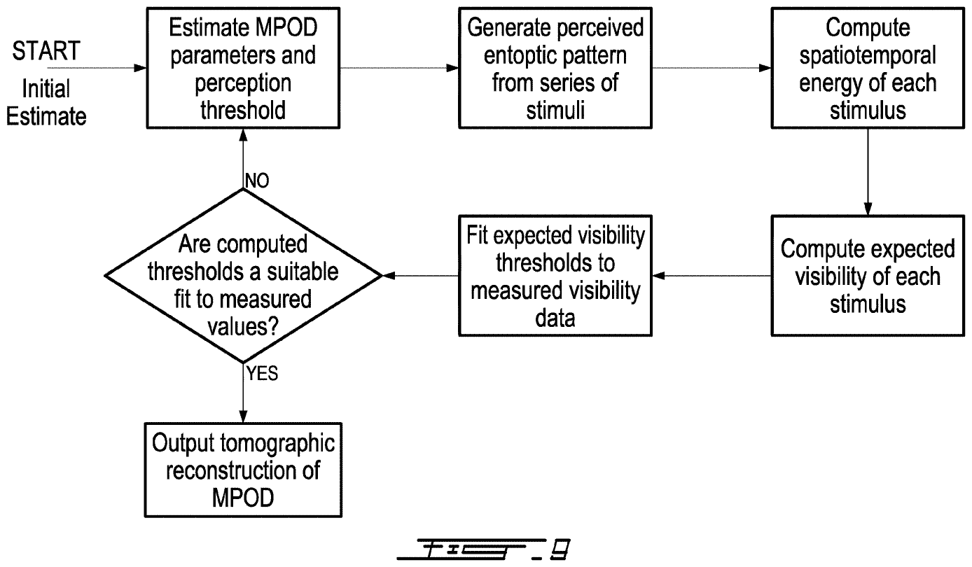

The method can be used in conjunction with models of human motion perception to reconstruct the spatial profile of an individual's macular structure (i.e. MPOD, Henle's fiber layer, etc.). At some contrast, humans are no longer able to perceive motion in their visual field. Models of human perception can be used to reconstruct the macular pigment density profile of an individual based on their psychophysical test results related to perception of entoptic motion. An example block diagram of this procedure can be seen in FIG. 9

Indeed, another method of evaluating the macular health and structure of an individual combines the structured light state of a particular stimulus with the MPOD of a particular individual. FIG. 9 depicts a flow chart of one possible tomographic reconstruction technique used to measure the MPOD of an individual based on the threshold visibility of polarization patterns. The initial estimate of MPOD parameters can use published averages of MPOD as measured by a variety of methods. From these parameters, and the structured light state of each structured state stimuli used in a series of experiments, the perceived entoptic pattern can be generated. From these entoptic patterns, the spatiotemporal energy can be computed by a variety of methods. Using this series of spatiotemporal energy values and the estimated energy threshold values, the threshold visibility of each entoptic pattern can be computed. These threshold entoptic visibilities can then be compared to measured entoptic threshold visibilities. The ‘goodness’ of this fit is then evaluated. If this fit is deemed ‘acceptable’, the MPOD parameters have successfully been reconstructed. If this fit is deemed ‘unacceptable’, the MPOD and spatiotemporal energy parameters are updated, and the reconstruction process is restarted.

One possible method of evaluating the macular health and structure of an individual eye is through the comparison of an individual's perception of entoptic patterns generated by a structured light state with stimuli generated with intensity-varying stimuli. The comparison could be performed using an intensity-varying stimuli pattern that matches the entoptic pattern generated by the structured light state. This comparison could be used to measure the visibility of entoptic patterns generated by structured light states.

The method can determine the contrast sensitivity of individuals to entoptic profiles. For instance, instead of measuring a threshold value of a parameter, one can subject the subject to two slightly different polarization profiles and request feedback from the subject as to whether or not the subject perceives a difference between the two. In a more elaborate form, the difference between the two different polarization profiles can be increased iteratively until the subject indicates that he/she perceives the contrast, which can provide a measure of the degree of difference in the parameter value of the polarization profile which corresponds to the sensitivity of the subject to contrast. In an even more elaborate form, the iterative process can continue by changing directions for the value of the parameter at each change in the subject's feedback until the process is deemed to have converged.

Moreover, the maximum degree of polarization can be used as a parameter in the methods discussed above. For instance, the threshold c could be measured for different degrees of polarization of the structured light, e.g. for a case where the structured light is 50% polarized and another one where the structured light is 100% polarized, which may lead to two different values of c, and provide insights into the macular behavior of the subject. Similarly, a value of c could be maintained constant while the degree of polarization is changed from one iteration to another, to find a threshold value of polarization at which a user can perceive the entoptic pattern with that particular value of c.

On another note, as the human eye ages, its sensitivity to faint patterns and motion may decrease. By measuring test results from un-structured light (i.e. an ordinary intensity pattern) the contrast sensitivity of an individual can be measured.

The method can be used in conjunction with a psychophysical task utilizing intensity-varying stimuli to distinguish changes in macular health and structure from changes in photoreceptor function.

For instance, in one example method, in a first step a subject can be subjected to a first one of structured light and normal light, and in a second step, the subject can be subjected to a second one of the structure light and normal light. The structured light can have a polarization profile configured to generate an entoptic profile for the subject, whereas the normal light can an intensity profile. There can be a geometrical correlation between the intensity profile and the entoptic profile. Accordingly, confirming that the subject can perceive the intensity profile can allow to validate that if the subject does not perceive the entoptic profile, something is amiss specifically in relation with the polarization perception of that subject.

Indeed, an individual's ability to perceive the motion of entoptic patterns generated by structured light may be inhibited by several factors including, changes in macular pigment density, changes in Henle's fiber layer, and changes in the photoreceptors which detect light. By comparing results from intensity-varying stimuli to results using structured light, changes in macular health can be distinguished from other causes of vision loss or degeneration, including changes in the photoreceptors.

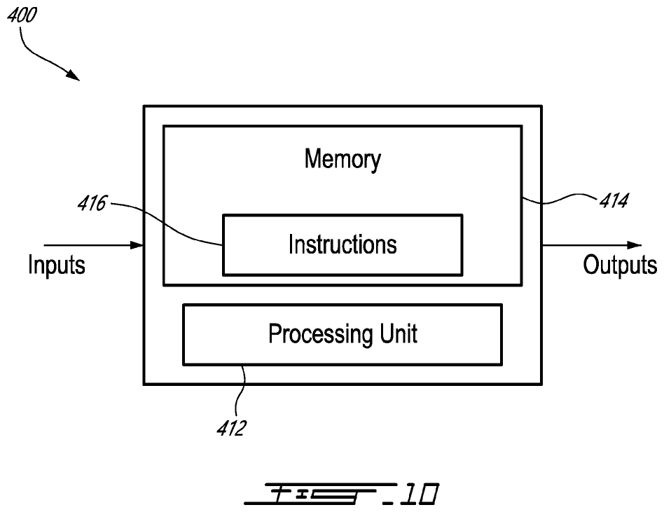

Referring to FIG. 10 it will be understood that the expression “computer” 400 as used herein is not to be interpreted in a limiting manner. It is rather used in a broad sense to generally refer to the combination of some form of one or more processing units 412 and some form of memory system 414 accessible by the processing unit(s). The memory system can be of the non-transitory type. The use of the expression “computer” in its singular form as used herein includes within its scope the combination of a two or more computers working collaboratively to perform a given function. Moreover, the expression “computer” as used herein includes within its scope the use of partial capabilities of a given processing unit.

A processing unit can be embodied in the form of a general-purpose micro-processor or microcontroller, a digital signal processing (DSP) processor, an integrated circuit, a field programmable gate array (FPGA), a reconfigurable processor, and a programmable read-only memory (PROM, to name a few examples.

The memory system can include a suitable combination of any suitable type of computer-readable memory located either internally, externally, and accessible by the processor in a wired or wireless manner, either directly or over a network such as the Internet. A computer-readable memory can be embodied in the form of random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) to name a few examples.

A computer can have one or more input/output (I/O) interface to allow communication with a human user and/or with another computer via an associated input, output, or input/output device such as a keyboard, a mouse, a touchscreen, an antenna, a port, etc. Each I/O interface can enable the computer to communicate and/or exchange data with other components, to access and connect to network resources, to serve applications, and/or perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, Bluetooth, WiMAX), SS7 signaling network, fixed line, local area network, wide area network, to name a few examples.

It will be understood that a computer can perform functions or processes via hardware or a combination of both hardware and software. For example, hardware can include logic gates included as part of a silicon chip of a processor. Software (e.g. application, process) can be in the form of data such as computer-readable instructions stored in a non-transitory computer-readable memory accessible by one or more processing units. With respect to a computer or a processing unit, the expression “configured to” relates to the presence of hardware or a combination of hardware and software which is operable to perform the associated functions. Different elements of a computer, such as processor and/or memory, can be local, or in part or in whole remote and/or distributed and/or virtual.

The methods and systems of the present disclosure may be implemented in a high level procedural or object oriented programming or scripting language, or a combination thereof, to communicate with or assist in the operation of a computer system, for example the controller 31. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 402 of the computing device 400, to operate in a specific and predefined manner to perform the functions described herein.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments. The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

As can be understood, the examples described above and illustrated are intended to be exemplary only. The scope is indicated by the appended claims.

What is claimed is:

1. A method comprising:
generating structured light having a polarization profile at a state-preparation plane; and
imaging the polarization profile of the structured light from the state-preparation plane to a retina of a subject.

2. The method of claim 1 wherein the polarization profile is a first polarization profile, the method further comprising: at a computer, collecting feedback from the subject following said imaging, selecting a second polarization profile based on said collected feedback, and repeating the steps of generating and imaging with the second polarization profile.

3. The method of claim 2 further comprising repeating the steps of generating, imaging, collecting, and selecting over a plurality of iterations, for a plurality of different polarization profiles, where a value of a parameter of the different polarization profiles changes from iteration to the next, including, at each repeating, comparing the subject's feedback to the subject's feedback from the previous iteration, until the comparison yields a change.

4. The method of claim 3 further comprising, when the comparison yields a change, outputting a current value of the parameter as a limit value of the parameter for the subject.

5. The method of claim 3 wherein the different values of the parameter associated to different iterations increase or decrease from one iteration to the next, further comprising, when the comparison yields a change, changing the value of the parameter to the other one of said increase or decrease, and resuming said repeating.

6. The method of claim 5 further comprising outputting a current value of the parameter as a limit value of the parameter for the subject when the iterations are deemed to have converged.

7. The method of claim 5 further comprising outputting a current value of the parameter as a contrast sensitivity value of the parameter for the subject when the iterations are deemed to have converged.

8. The method of claim 3 wherein the parameter is one of size of obstruction, form of obstruction, number of lines, density of azimuthal lines, rotation speed, and degree of polarization.

9. The method of claim 1 wherein said generating structured light includes generating a fixation point.

10. The method of claim 1 wherein the polarization profile includes at least three lines, preferably between 10 and 30 lines.

11. The method of claim 10 wherein the lines are azimuthally distributed.

12. The method of claim 10 wherein said generating structured light includes rotating the polarization profile over time.

13. The method of claim 1 further comprising acquiring an image of the image of the polarization profile at the retina.

14. The method of claim 1 further comprising, exposing the retina of the subject to light having an intensity profile, the intensity profile having a geometrical correlation with the polarization profile.

15. A system comprising:
a structured light generator configured for generating structured light having a polarization profile at a state-preparation plane; and
an ocular imaging assembly configured for imaging the polarization profile from the state-preparation plane to a retina of a subject.

16. The system of claim 15 further comprising a camera optically coupled to the retina via the ocular imaging assembly.
